(19) United States
(12) Patent Application Publication
(10) Pub. No.: US 2012/0100561 A1
(43) Pub. Date: Apr. 26, 2012

(54) A PKN3/RHOC MACROMOLECULAR COMPLEX AND METHODS OF USE THEREFOR

(76) Inventors: Anke Klippel-Giese, Park Ridge, NJ (US); Keziban Unsal-Kacmaz, Park Ridge, NJ (US)

(21) Appl. No.: 13/256,207

(22) PCT Filed: Mar. 12, 2010
(86) PCT No.: PCT/US10/27095

§ 371 Date: Nov. 29, 2011

Related U.S. Application Data
(60) Provisional application No. 61/159,739, filed on Mar. 12, 2009.
(60) Provisional application No. 61/226,078, filed on Jul. 16, 2009.

(51) Int. Cl. G01N 33/574; C07K 2/00; C07K 14/47; G01N 33/573

(52) U.S. Cl. 435/7.23; 435/7.21; 530/300; 530/324

(57) ABSTRACT

Disclosed are compositions comprising and methods of using a novel macromolecular assembly comprising PKN3, PDK1 and RhoC (PPRC complex). The PPRC complex was shown to have kinase activity and was found in cells of high malignancy potential, such as particularly aggressive cancers. In some aspects, the invention provides methods for screening compounds that have cancer therapeutic potential, methods for diagnosing aggressive cancer, methods for determining the prognosis of a patient suffering from cancer, methods for stratifying patients in a clinical trial or determining the effectiveness of a particular treatment regimen, polypeptides that modulate the formation of the PPRC complex, and kits comprising one or more components of the PPRC complex.

### BACKGROUND

The present invention is related to the use of protein kinase N 3 (PKN3) and RhoC to screen compounds for anti-cancer activity and to diagnose patients having aggressive cancer.

The development of effective cancer therapies increasingly relies on the elucidation of the molecular mechanisms underlying the disease, and the identification of target molecules within those mechanisms which may be useful in the development of new drugs. Once such target molecules are available, drug candidate compounds can be tested against those targets. In many cases, such drug candidates are members of a compound library which may consist of synthetic or natural compounds.

There is significant need to identify new molecular targets associated with particularly aggressive forms of cancer so that new therapeutic compounds and regimens can be identified and validated.

### SUMMARY

In a first aspect, the invention provides a method of identifying compounds that are useful in the treatment of cancer. The steps of this method include combining a test compound with a protein kinase N3 (PKN3) polypeptide (or fragment thereof), a RhoC polypeptide (or fragment thereof), and a phosphoinositide-dependent kinase-1 (PDK1) polypeptide (or a fragment thereof), and then determining the amount of a complex containing PKN3 and RhoC (“complex”) that is formed. This is called the “test level” of the complex. The test level is then compared to a reference. Any difference between the test level and the reference indicates that the test compound potentially has cancer therapeutic activity. In some embodiments, the PKN3, RhoC and PDK1 polypeptides (or fragments thereof) are purified to an extent prior to combining with the test compound.

In some embodiments, one or more of the components (the components being PKN3, RhoC and PDK1, or respective fragments thereof) contain a molecular tag used to identify the component and to aid in the isolation of the component and any other components attached thereto. Molecular tags are generally known in the art. Examples include polyhistidine, GST and FLAG. In some embodiments, the PKN3 polypeptide comprises a molecular tag and the complex is isolated by pulling down the PKN3 polypeptide by its molecular tag.

In some embodiments, the reference is a level of a complex that is formed in the absence of the test compound. In other embodiments, the reference is the level of a complex that is formed when the test compound is combined with a PKN3, RhoC and PDK1 (or respective fragments thereof), wherein any one or both of the kinases are kinase-dead.

In some embodiments, the test level is less than the reference. In some embodiments, the test compound is a compound that decreases the invasiveness of a cancer cell or decreases the rate of growth of a tumor.

In a second aspect, the invention provides a cell-based method of identifying compounds that are useful in the treatment of cancer. The steps of this method include contacting a cell with a test compound, and then determining the amount of a complex that is formed in the cell in the presence of the test compound (test level). The cell contains a PKN3 polypeptide (or fragment thereof), a RhoC polypeptide (or fragment thereof), and a phosphoinositide-dependent kinase-1 (PDK1) polypeptide, and the complex contains each of those components as well. The test level is then compared to a reference. Any difference between the test level and the reference indicates that the test compound potentially has cancer therapeutic activity.

In some embodiments, the cell is a stem cell, such as, e.g., a long term hematopoietic stem cell (LT-HSC). In other embodiments, the cell is a cell cultured from a tumor. In some embodiments, the cell has a high potential for metastasis. In some embodiments, the cell is one of, e.g., PC3 cell, HEK293 cell, MDA-MB231 cell, MCF7 cell, MDA361 cell, MCF468 cell, BT549 cell and HeLa cell. In other embodiments, the cell contains one or more or all of the components as heterologous recombinant polypeptides.

In some embodiments, one or more of the components (the components being PKN3, RhoC and PDK1 or fragments thereof) are endogenous to the cell. In some embodiments, one or more of the components are recombinant and contain a molecular tag used to identify the component and to aid in the isolation of the component and any other components attached thereto. Molecular tags are generally known in the art. Examples include polyhistidine, GST and FLAG. In some embodiments, the PKN3 polypeptide comprises a molecular tag and the complex is isolated by pulling down the PKN3 polypeptide by its molecular tag.

In some embodiments, the reference is a level of a complex that is formed in the absence of the test compound. In other embodiments, the reference is the level of a complex that is formed when the test compound is combined with a PKN3, RhoC and PDK1 (or fragment thereof), wherein any one or both of the kinases are kinase-dead.

In some embodiments, the test level is less than the reference. In some embodiments, the test compound is a compound that decreases the invasiveness of a cancer cell or decreases the rate of growth of a primary tumor.

In a third aspect, the invention provides a method of diagnosing cancer in a patient by assessing the levels of PKN3 activity and RhoC activity in the patient. According to the method, a sample is obtained from the patient, then the levels of PKN3 activity and RhoC activity in the sample are determined (this is the test level), and then the test level is compared to a reference. A difference between the test level and the reference indicates that the patient has a cancer that is aggressive.

In a fourth aspect, the invention provides a method of identifying a patient who can respond to a cancer therapy by assessing the levels of PKN3 activity and RhoC activity in the patient. According to the method, a sample is obtained from the patient, then the levels of PKN3 activity and RhoC activity in the sample are determined (this is the test level), and then the test level is compared to a reference. A difference between the test level and the reference indicates the likelihood of whether the patient will respond to the cancer therapy.

In some embodiments of both the third aspect and the fourth aspect, the reference is the level of PKN3 activity and RhoC activity determined in a non-cancer tissue. In some embodiments, the sample is a tumor biopsy and the test level is determined by an in situ assay in the tumor, such as, e.g., an in situ hybridization, in situ PCR or immunostaining. In some embodiments, the reference is simply any non-cancer tissue surrounding or within the tumor sample. In some embodiments, the test level is greater than the reference.

In a fifth aspect, the invention provides a method for determining the efficacy of a cancer therapeutic treatment regimen in a subject by assessing the change in the levels of activities of PKN3 and RhoC before administering the treatment and after administering the treatment. According to the method, a sample is obtained from the patient, then the levels of PKN3 activity and RhoC activity in the sample are determined (this is the pre-therapy or first level,) then a treatment regimen is administered to the patient. At a time after the treatment is administered, a second sample is obtained from the patient, and the levels of PKN3 activity and RhoC activity in the second sample are determined (this is the post-therapy or second level). The first and second levels are compared to each other. A decrease in both PKN3 activity and RhoC activity in the second level relative to the first level indicates that the treatment regimen is effective against a cancer in the patient.

In some embodiments of the third, fourth and fifth aspects, the PKN3 activity are the expression of an RNA that encodes a PKN3 polypeptide or fragment thereof. In other embodiments, the PKN3 activity is the expression of a PKN3 polypeptide or fragment thereof. In other embodiments, PKN3 activity is the phosphorylation of a PKN3 polypeptide. In still other embodiments, the PKN3 activity is the phosphorylation of a downstream effector of PKN3, such as, e.g., a glycogen synthase kinase 3 (GSK-3)-derived peptide (see also Table 1.)

In some embodiments of the third, fourth and fifth aspects, the RhoC activity are the expression of an RNA that encodes a RhoC polypeptide or fragment thereof. In other embodiments, the RhoC activity is the expression of a RhoC polypeptide or fragment thereof. In other embodiments, RhoC activity is the phosphorylation of a downstream effector of RhoC, such as, e.g., PKN3 or a glycogen synthase kinase 3 (GSK-3)-derived peptide (see also Table 1.)

In some embodiments of the third, fourth and fifth aspect, the phosphorylation status of the PKN3 polypeptide is determined by using an antibody or other polypeptide that specifically binds to a turn motif phosphorylation site at T860 of PKN3. See, e.g., SEQ ID NO.: 36.

In some embodiments of the first through fifth aspects, the cancer is any one or more of breast cancer, ovarian cancer, pancreatic cancer, gastric cancer, hepatocellular carcinoma, bladder cancer, colorectal cancer, cutaneous melanoma and carcinoma of the prostate (CaP).

In a sixth aspect, the invention provides a composition that comprises a polypeptide or peptidomimetic that disrupts or blocks the formation of a complex comprising a PKN3 polypeptide and RhoC polypeptide. In some embodiments, the polypeptide or peptidomimetic binds to a region of PKN3 that binds to RhoC. In other embodiments, the polypeptide or peptidomimetic binds to a region of RhoC that binds to PKN3. In some embodiments, the polypeptide or peptidomimetic contains one or more complementarity determining regions (CDR), which recognize an epitope on PKN3 or RhoC. In some embodiments, the CDR-containing polypeptide is any one of an antibody, a monoclonal antibody, a humanized antibody, a single chain single chain variable fragments (“ScFv”), a small modular immunopharmaceutical (“SMIP”), and a nanobody (a.k.a. single domain antibodies or VHH antibodies.)

In some embodiments of the sixth aspect, the region of PKN3 that binds to RhoC comprises any one or more of an ACC1, ACC2 or ACC3 amino acid sequence. In other embodiments, the polypeptide or peptidomimetic contains an amino acid sequence corresponding to any one or more of ACC1, ACC2 and ACC3 of a PKN3.

In a seventh aspect, the invention provides a polypeptide that contains at least one complementarity determining region (CDR) that recognizes a PKN3 turn motif phosphorylation site at T860. In some embodiments, the polypeptide is an antibody, which include, e.g., a monoclonal antibody, a humanized antibody, a single chain single chain variable fragment (“ScFv”), a small modular immunopharmaceutical (“SMIP”), and a nanobody (a.k.a. single domain antibodies or VHH antibodies.).

In an eighth aspect, the invention provides a kit that is used to screen for compounds that modulate the interaction between PKN3 and RhoC, and which can be used to treat cancer. The kit can also be used to determine the aggressiveness or invasiveness of a cancer in a patient. The kit can also be used to assess the effectiveness of cancer therapy in a patient. In some embodiments, the kit contains (a) an agent that detects a PKN3 activity, (b) an agent that detects a RhoC activity, (c) a label and (d) a package. In some embodiments, the kit also contains a cancer therapeutic compound. A cancer therapeutic compound is a compound, composition or treatment regimen that prevents or delays the growth or metastasis of cancer cells in a subject. Such cancer therapeutic compounds include, but are not limited to, chemotherapeutic drugs, gene therapy compositions, compounds that affect hormones, immunotherapeutic compounds, antibodies and antisense oligonucleotides. Examples of useful chemotherapeutic drugs include, but are not limited to, bleomycin, neocarcinostatin, suramin, doxorubicin, taxol, mitomycin C and cisplatin. It is to be understood that cancer therapeutic compounds for use in the present invention also include novel compounds or treatments developed in the future.

### DRAWINGS

FIG. 1 depicts a Western blot of breast cancer cell lines having increasing malignant potential showing concomitantly increasing levels of phosphorylated PKN3.

FIG. 2 depicts a Western blot of non-small cell lung carcinoma (NSCLC) cell lines having increasing drug resistance showing concomitantly increasing levels of phosphorylated PKN3.

FIG. 3, panel A depicts a Western blot of lysates from cells transfected with Myc-tagged Rho and Rac constructs probed with anti-phosphorylated PKN and anti-Myc antibodies. Panel B depicts a Western blot of anti-Myc immunoprecipitates from cells transfected with Myc-tagged Rho and Rac constructs probed with anti-phosphorylated PKN and anti-Myc antibodies.

FIG. 4, panel A depicts a Western blot of lysates from cells transfected with Myc-tagged Rho constructs and Flag-tagged PKN3 constructs probed with anti-Myc and anti-Flag antibodies. Panel B depicts a Western blot of an anti-Flag immunoprecipitation that shows the kinase-dependent formation of a ternary complex containing PKN3, RhoC and PDK1.

FIG. 5, panel A depicts a Western blot of lysates from cells transfected with Myc-tagged Rho constructs and Flag-tagged PKN3 constructs probed with anti-Myc, anti-PDK1, and anti-PKN3 antibodies. Panel B depicts a Western blot of an anti-Flag immunoprecipitation depicting the kinase activity of PKN3 regulated by the RhoC/PDK1/PKN3 ternary complex.

FIG. 6, panel A depicts a Western blot of lysates from PC-3 cells expressing doxycycline (Dox or Doxy)-induced shRNAs that target PKN3 or p110β. Panel B depicts representative cell populations that were seeded on MATRIGEL®.

FIG. 7, panels A and B depict histograms quantifying tumor volume from mice harboring transplanted PKN3 shRNA PC-3 cells.

FIG. 8, panel A depicts a Western blot of lysates from MDA-MB-231 cells expressing Dox-induced shRNAs that target PKN3, p110β or CKIε. Panel B depicts representative cell populations that were seeded on MATRIGEL®.

FIG. 9 depicts a scatter plot showing tumor mass in mice harboring PKN3 shRNA MDA-MB-231 cells, with and without doxycyclin induction.

### DETAILED DESCRIPTION

Disclosed herein is the surprising discovery that (a) protein kinase N3 (PKN3) preferentially associates with RhoC, (b) PKN3 binds to RhoC in a kinase-dependent manner, and (c) the association of PKN3 and RhoC facilitates the formation of a ternary complex containing PDK1 (PKN3/PDK1/RhoC complex or PPRC complex). The PPRC complex is a valuable target in connection with particularly aggressive cancers. It is further disclosed that the formation of a PPRC complex results in the increased phosphorylation of PKN3 and its subsequent kinase activity.

PKN3 is a serine/threonine protein kinase of 889 amino acid residues in length (human orthologue). It has an N-terminal putative regulatory region containing three antiparallel coiled-coil (ACC) domains ACC1, ACC2 and ACC3 located at about residues 15-77, 97-170 and 184-236, respectively; a C-terminal catalytic region located at residues 559-882; and a C2-like domain of about 100 to 130 residues in length positioned between the putative regulatory domain and the catalytic domain. There are at least three different isoforms of PKN (PKN1/PKNα/PAK-1/PRK-1, PKN2/PRK2/PAK-2/PKNγ, and PKN3/PKNβ) in mammals, each of which shows different enzymological properties, tissue distribution, and varied functions. For a review of PKN, see Mukai, H., J. Biochem. 133:17-27, 2003. See also U.S. Patent Application No: 20040106569, published Jun. 3, 2004, which is incorporated herein by reference in its entirety.

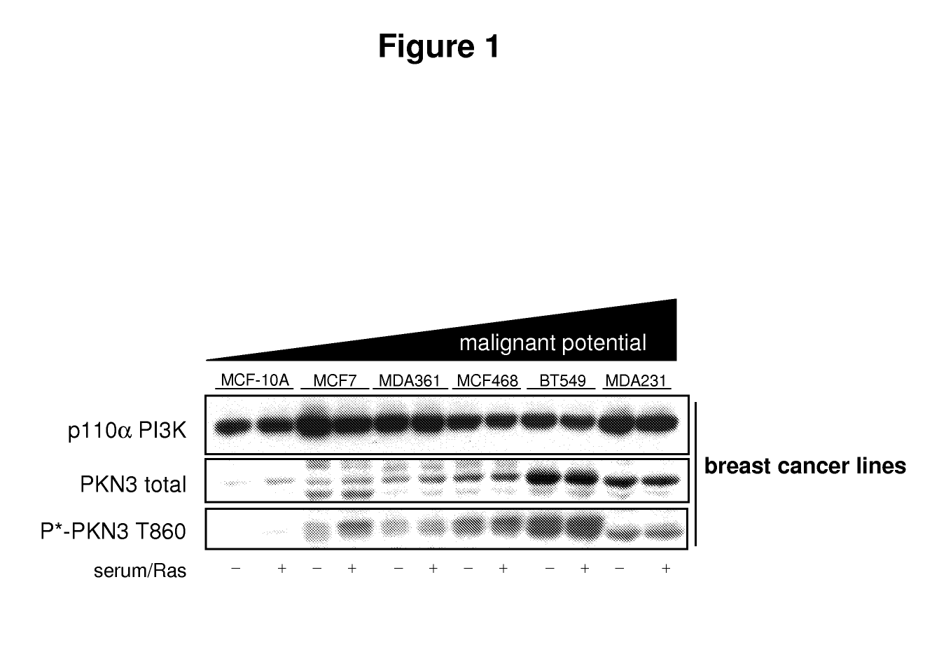

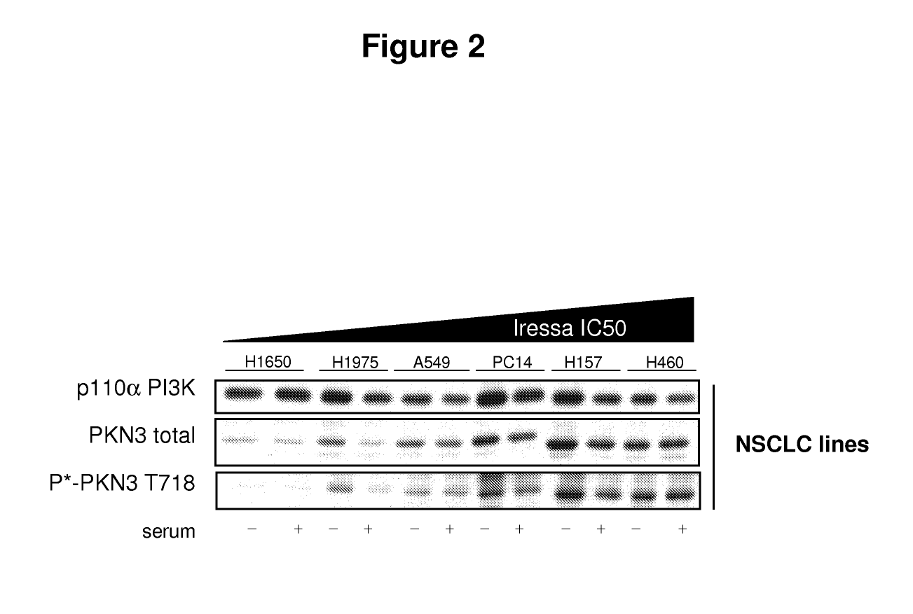

It is further disclosed herein that PKN3 is up-regulated in cancer cells having increased aggressiveness and drug resistance (see FIGS. 1 and 2, respectively). By increased aggressiveness, what is meant is that the cancer cells are metastatic, have high potential to metastasize, have increased rate of proliferation, or are drug resistant. An aggressive cancer is exemplified by, e.g., a triple-negative breast cancer (see, e.g., Dent et al., Clinical Cancer Research 13: 4429-4434, Aug. 1, 2007). Aggressive cancers also comprise those cancers in which the PKN3/RhoC pathway is involved.

Compounds that inhibit the activity of the PPRC complex can be used to control metastatic and proliferational behavior of cells and therefore provide methods of treating tumors and cancers, more particularly those tumors and cancers which are aggressive. The reduction in signaling and other activities that are effected by PPRC activity may stem either from a reduction at the transcription level, at the level of the translation, or at the level of post-translational modification of one or more of the PPRC complex components, or at the level of quaternary structure formation (i.e., formation of a ternary complex).

Because of the involvement of the PPRC complex and the RhoC/PKN3 pathway in aggressive cancer, the complex and its components can be used as a prognostic marker, a disease staging marker, a patient-stratification marker, or a marker for diagnosing the status of a cell or patient having in his body such kind of cells as to whether the cell will undergo metastasis or otherwise become aggressive.

PKN3 is a developmentally regulated mediator of PI3K-induced migration and invasion of cells. It is regulated by PI3K at the level of expression and catalytic activity in an Akt-independent manner. It has a restricted expression pattern (endothelial, embryonic and tumor cells) and is not essential for most normal cell function. It is required for metastatic PC-3 (PTEN−/−) cell growth in an orthotopic mouse model.

In normal cells, the PI3-kinase (phosphatidyl-inositol-3-kinase) pathway is characterized by a PI3-kinase activity upon growth factor induction and a parallel signaling pathway. Growth factor stimulation of cells leads to activation of their cognate receptors at the cell membrane which in turn associate with and activate intracellular signaling molecules such as PI3-kinase. Activation of PI3-kinase (consisting of a regulatory p85 and a catalytic p110 subunit) results in activation of Akt by phosphorylation, thereby supporting cellular responses such as proliferation, survival or migration further downstream. PTEN is thus a tumor suppressor which is involved in the phosphatidylinositol (PI) 3-kinase pathway and which has been extensively studied in the past for its role in regulating cell growth and transformation (for reviews, see, e.g., Stein, R. C. and Waterfield, M. D. Mol Med Today 6:347-357, 2000).

The tumor suppressor PTEN functions as a negative regulator of PI3-kinase by reversing the PI3-kinase-catalyzed reaction and thereby ensures that activation of the pathway occurs in a transient and controlled manner. Chronic hyperactivation of PI3-kinase signaling is caused by functional inactivation of PTEN. PI3-kinase activity can be blocked by addition of the small molecule inhibitor LY294002. The activity and downstream responses of the signaling kinase MEK which acts in a parallel pathway, can, for example, be inhibited by the small molecule inhibitor PD98059.

Chronic activation of the PI3-kinase pathway through loss of PTEN function is a major contributor to tumorigenesis and metastasis, indicating that this tumor suppressor represents an important checkpoint for a controlled cell proliferation. PTEN knock-out cells show similar characteristics as those cells in which the PI3-kinase pathway has been chronically induced via activated forms of PI3-kinase. Activation of phosphatidylinositol 3-kinase is sufficient for cell cycle entry and promotes cellular changes characteristic of oncogenic transformation.

Diseases and conditions involving dysregulation of the PI3-kinase pathway are well known. Any of these conditions and diseases may thus be addressed by the inventive methods and the drugs and diagnostic agents, the design, screening or manufacture thereof is taught herein. For reasons of illustration but not limitation conditions and diseases are referred to the following: endometrial cancer, colorectal carcinomas, gliomas, endometrial cancers, adenocarcinomas, endometrial hyperplasias, Cowden's syndrome, hereditary non-polyposis colorectal carcinoma, Li-Fraumene's syndrome, breast cancer, ovarian cancer, prostate cancer, Bannayan-Zonana syndrome, LDD (Lhermitte-Duklos' syndrome), hamartoma-macrocephaly diseases including Cow disease (CD) and Bannayan-Ruvalcaba-Rily syndrome (BRR), mucocutaneous lesions (e.g., trichilemmonmas), macrocephaly, mental retardation, gastrointestinal harmatomas, lipomas, thyroid adenomas, fibrocystic disease of the breast, cerebellar dysplastic gangliocytoma and breast and thyroid malignancies.

In view of this, the PPRC complex and its individual components are valuable downstream drug targets of the PI3-kinase pathway which can be addressed by drugs which will have less side effects than other drugs directed to targets upstream of PPRC. Thus, the present invention provides a drug target which is suitable for the design, screening, development and manufacture of pharmaceutically active compounds which are more selective than those known in the art, such as, for example, 2-(4-morpholinyl)-8-phenylchromone (“LY 294002”), which generally target PI3-kinase. By having control over this particular fraction of effector molecules, i.e., the RhoC and PKN3 and any further downstream molecule involved in the pathway, only a very limited number of parallel branches thereof or further upstream targets in the signaling cascade are likely to cause unwanted effects. Therefore, the other activities of the PI-3 kinase/PTEN pathway related to cell cycle, DNA repair, apoptosis, glucose transport, translation will not be influenced. Also, the insulin signaling is not induced, which means that the diabetic responses or other side effects observed in connection with the use of LY294002 are actually avoided.

The complete sequence of a nucleic acid encoding PKN3 is generally available in databanks, e.g., under accession number NM_{—}013355.3. Also, the amino acid sequence of PKN3 is available in databanks under the accession number NP_{—}037487.2. The complete sequence of a nucleic acid encoding RhoC is generally available in databanks, e.g., under accession numbers NM_{—}001042678.1, NM_{—}001042679.1 and NM_{—}175744.4. Also, the amino acid sequence of RhoC is available in databanks under the accession numbers NP_{—}001036143.1, NP_{—}001036144.1 and NP_{—}786886.1. It is within the present invention that PKN3 and RhoC derivatives or truncated versions thereof may be used according to the present invention as long as the desired effects may be realized. The extent of derivatization and truncation can thus be determined by one skilled in the art by routine analysis.

In the context of the present invention, the term nucleic acid sequences encoding PKN3 and RhoC also include nucleic acids which hybridize to nucleic acid sequences specified by the aforementioned accession numbers or any nucleic acid sequence which may be derived from the aforementioned amino acid sequences. Such hybridization is known to the skilled artisan. The particularities of such hybridization may be taken from Sambrook, J. Fritsch, E. F. and Maniatis, T. (1989) Molecular Cloning: A Laboratory Manual, 2nd ed. Cold Spring Harbor: Cold Spring Harbor Laboratory. In a preferred embodiment the hybridization is a hybridization under stringent conditions, for example, under the stringent conditions specified in Sambrook supra.

In addition, nucleic acids encoding a PKN3 and RhoC are also nucleic acid sequences which contain sequences homologous to any of the aforementioned nucleic acid sequences, whereby the degree of sequence homology is 75, 80, 85, 90 or 95%.

Orthologues to human PKN3 may be found, among others, in organisms as evolutionarily diverse as M. musculus and R. norvegicus, A. thaliana, C. elegans, D. melanogaster and S. cerevisiae. In the case of PKN3, the percent identity is 67%, 51%, 38%, 36%, 63% and 44%, respectively, for the various species mentioned before. Orthologues to human RhoC are found in mouse, rat, arabidopsis and drosophila, with percent identities of 100%, 99%, 47% and 86%, respectively. It will be acknowledged by the skilled artisan that any of these or other orthologues and homologues will in principle be suitable for the practice of the present invention, provided the drug or diagnostic agent generated using such homologue may still interact with the human PKN3 or RhoC or any other intended PKN3 or RhoC.

The PPRC complex and individual members thereof may be used as a target against which chemical compounds, which may be used as drugs or drug candidates or as diagnostic agents, are directed. Suitable chemical compounds belonging to different classes of compounds such as antibodies, peptides, anticalines, aptamers, spiegelmers, ribozymes, antisense oligonucleotides and siRNA, as well as small organic molecules, may be used. The compounds are designed, selected, screened generated or manufactured by either using the PPRC complex (the complex itself, individual members thereof or combinations thereof) or information related to the PPRC complex.

In the design, selection, screening, generation or manufacturing process of said classes of compounds, PPRC will also be referred to as the target which is used in the process rather than in the final application of the respective compound to a patient in need thereof. In the processes which provide the various classes of compounds, either the entire PPRC complex, individual members or various combinations thereof, or nucleic acids encoding for any and all of the protein constituents of the PPRC may be used. The term “PPRC complex or components thereof” as used herein comprises any fragment or derivative of PPRC and its constituent members, which allows the design, selection, screening, generation or manufacture of said compound which can be used as a medicament or as a diagnostic agent.

The term “nucleic acid encoding the PPRC complex or components thereof” as used herein shall comprise any nucleic acid, which contains a nucleic acid that encodes any component of the PPRC complex or fragment thereof. A part of a nucleic acid encoding the PPRC complex or components thereof is regarded as such as long as it is suitable for the design, selection, screening, generation or manufacture of said compound which can be used as a medicament or as a diagnostic agent. The nucleic acid encoding the PPRC complex or components thereof may be genomic nucleic acid, hnRNA, mRNA, cDNA or part of each thereof.

As outlined above, it is within the present invention that apart from the PPRC complex or components thereof or a nucleic acid sequence therefore, as described herein, other means or compounds may be used in order to create or to suppress the effects arising from the endogenous activity of the PPRC complex. Such means may be determined or selected in a screening method. In such screening method, a first step is to provide one or several so-called candidate or test compounds. Candidate compounds as used herein are compounds the suitability of which is to be tested in a test system for treating or alleviating cancer as described herein or to be used as a diagnostic means or agent for cancer.

If a candidate compound shows a respective effect in a test system, said candidate compound is a suitable means or agent for the treatment of said diseases and diseased conditions and, in principle, as well a suitable diagnostic agent for said diseases and diseased conditions. In a second step, the candidate compound is contacted with a PPRC expression system, a PPRC gene product, a PPRC activity system, or a PPRC complex or component. The PPRC activity system is also referred to herein as a system detecting the activity of the PPRC complex, such as, e.g., PPRC kinase activity. In some embodiments, the kinase activity of the PPRC complex can be assessed by determining the phosphorylation of a substrate, such as, e.g., a diagnostic GSK3α-derived fragment having a sequence of GPGRRGRRRTSSFAEGG (SEQ ID NO:1). Table 1 depicts additional PPRC kinase substrates useful in the practice of the invention.

The PPRC screening methodology described herein also is useful to eliminate non-functional or inactive compounds from further consideration. Thus PPRC activity can be measured in a first sample obtained from a subject or test system, generating a pre-treatment level, followed by administering a test compound to the subject or test system and measuring the activity of the PPRC complex or of individual components thereof in a second sample from the subject or test system at a time following administration of the test compound, thereby generating data for a test level. The pre-treatment level (first level) can be compared to the test level (second level), and data showing no decrease in the test level relative to the pre-treatment level indicates that the test compound is not effective in the subject, and the test agent may be eliminated from further evaluation or study. Conversely, a change in values can indicate that the test compound is suitable for use as a PPRC inhibitor or for further study.

TABLE 1
Phosphoproteins Modulated by PKN3

| SEQ ID | | |
|---|---|---|
| NO: | Protein Entry | |
| 2 | Serine/threonine-protein kinase | R.RAIPTVNHSGTFS*PQAPVPTTVPVVDVR.I |
| 3 | N2 | K.EGMGYGDRTST*FCGTPEFLAPEVLTETSYTR.A |
| 4 | Serine/threonine-protein kinase | R.RGPS*PPASPTR.K |
| 5 | N3 | K.GCPRT*PTTLR.E |
| 6 | | K.GCPRT*PTTLREASDPATPSNFLPK.K |
| 7 | | K.EGIGFGDRTST*FCGTPEFLAPEVLTQEAYTR.A |
| 8 | Pleckstrin homology domain-containing | K.ERPISMINEASNYNVTSDYAVHPMS*PVGR.T |
| | family A member 5 | |
| 9 | Abl interactor 1 | R.TNPPTQKPPS*PPMSGR.G |
| 10 | Coiled-coil domain-containing protein 16 | K.KEEENADS*DDEGELQDLLSQDWRVK.G |
| 11 | hypothetical protein LOC348262 | R.TSSPRS*PPSSSEIFTPAHEENVR.F |
| 12 | B-cell lymphoma 6 protein | K.FIVLNSLNQNAKPEGPEQAELGR\hat{ }LS*PR.A |
| 13 | Isoform DPII of Desmoplakin | K.GGGGYTCQS*GSGWDEFTK.H |
| 14 | 40S ribosomal protein S27-like protein | R.LTEGCS*FR.R |
| 15 | Fiibronectin | R.TNTNVNCPIECFMPLDVQADREDS*RE. |
| 16 | Tenascin | K.LPVGSQCSVDLESAS*GEK.D |
| 17 | Bone sialoprotein 2 | K.IEDS*EENGVFK.Y |
| 18 | FERM domain-containing protein 3 | K.AAREYEDPPS*EEEDK.I |
| 19 | Protocadherin alpha-4 | K.FIIPGS*PAIISIR.Q |
| 20 | triple functional domain | K.DSLSVSSNDASPPASVASLQPHMIGAQSS*PGPK.R |
| 21 | Protein phosphatase 1 regulatory subunit 12C | R.RST*QGVTLTDLKEAEK.A |
| 22 | Replication factor C subunit 5 | K.EFGSMVLELNASDDRGIDIIRGPILS*FASTR.T |
| 23 | Putative heat shock protein HSP 90-alpha A2 | K.ESKDKPEIEDVGS*DEEEEK.K |

A PPRC expression system is basically an expression system that shows or displays the expression of any one or more of the PPRC component polypeptides (RhoC, PKN3 and PDK1), whereby the extent or level of expression may be changed. A PPRC activity system is essentially an expression system whereby the activity or condition of activity (such as, e.g., kinase activity) is measured.

In any of these systems it is determined whether under the influence of a candidate compound the activity of PPRC or of the nucleic acids encoding PPRC is different from the situation without the candidate compound. Regardless whether the particular system is either an expression system or an activity system, it is within the scope of the present invention that either an increase or a decrease of the activity and expression, respectively, may occur and be measured. Typically, the expression system or activity system is an in vitro reaction, such as a cell extract or a fraction of the cell extract such as a nuclear extract. A PPRC expression system as used herein may also be a cell, preferably a cell of a tissue or organ involved in the diseases as described herein and diseased conditions as described herein.

Whether there is an increase or decrease in the activity system or expression system may be determined at each level of the expression, for example, by measuring the increase or decrease of the amount of nucleic acid coding for the PPRC complex or components thereof, more particularly mRNA, or the increase or decrease of the polypeptides of the PPRC complex or components thereof expressed under the influence of the candidate compound. The techniques required for such measurements, more particularly the quantitative measurement of these kinds of changes, such as for the mRNAs or the polypeptides, are known to the skilled artisan. Also known to the skilled artisan are methods to determine the amount of or content of the polypeptides of the PPRC complex, e.g., by detection using appropriate antibodies. Antibodies may be generated as known to the skilled artisan and described, e.g., by Harlow, E., and Lane, D., “Antibodies: A Laboratory Manual,” Cold Spring Harbor Laboratory, Cold Spring Harbor, N.Y., (1988). Suitable antibodies may also be generated by other well known methods, for example, by phage display selection from libraries of antibodies.

In case of a PPRC complex expression system, an increase or decrease of the activity of PPRC complex may be determined, preferably in a functional assay.

Contacting the candidate compound and the expression system and activity system, respectively, usually is performed by adding an aqueous solution of the candidate compound to a respective reaction system which is generally referred to herein as the test system. Besides aqueous solutions (which may be buffered), suspensions or solutions of the candidate compound in organic solvents or in mixtures of organic and aqueous solvents may be used.

In some embodiments, in each run using the expression system and activity system, respectively, only a single candidate compound is used. However, it is also within the present invention that several of this kind of tests is performed in parallel in a high throughput system using methods known in the art.

A further step in the method according to the present invention resides in determining whether under the influence of the candidate compound the expression or activity of the expression system and activity system, respectively, in relation to the PPRC complex or a nucleic acid coding therefore is changed. Typically this is done by comparing the system's reaction upon addition of the candidate (test) compound relative to the one without addition of the candidate compound. Alternatively, this can be done by comparing the system's reaction upon addition of the candidate compound relative to the reaction of a system containing a non-functional PPRC component, e.g., containing a kinase-dead PKN3, upon the addition of the candidate compound. In some embodiments, the candidate compound is a member of a library of compounds.

Generally, any library of compounds is suitable for the purpose of this invention regardless of the class of compounds. Suitable libraries of compounds are, among others, libraries composed of small molecules, peptides, proteins, antibodies, or functional nucleic acids. The latter compounds may be generated as known to the skilled artisan.

The manufacture of an antibody, which is specific for the PPRC complex as a whole or any component or combination of components thereof or their fragments, is known to the skilled artisan. The antibodies of the invention include nanobodies, polyclonal antibodies, monoclonal antibodies, chimeric antibodies (e.g., humanized antibodies), and anti-idiotypic antibodies. Polyclonal antibodies are heterogeneous populations of antibody molecules derived from the sera of animals immunized with an antigen. Monoclonal antibodies are a substantially homogeneous population of antibodies that bind to specific antigens. In general, antibodies can be made, for example, using traditional hybridoma techniques (Kohler and Milstein (1975) Nature, 256: 495-499), recombinant DNA methods (U.S. Pat. No. 4,816,567), or phage display using antibody libraries (Clackson et al. (1991) Nature, 352: 624-628; Marks et al. (1991) J. Mol. Biol., 222: 581-597). For additional antibody production techniques, see Antibodies: A Laboratory Manual, eds. Harlow and Lane, Cold Spring Harbor Laboratory, 1988. The present invention is not limited to any particular source, method of production, or other special characteristics of an antibody.

The term “antibody” is also meant to include both intact molecules as well as fragments such as Fab, single chain Fv antibodies (ScFv) and small modular immunopharmaceuticals (SMIPs), which are capable of binding antigen. Fab fragments lack the Fc fragment of intact antibody, clear more rapidly from the circulation, and may have less non-specific tissue binding than an intact antibody (Wahl et al., 1983, J. Nucl. Med. 24:316-325). Chimeric antibodies are molecules, different portions of which are derived from different animal species, such as those having variable region (VH, VL) derived from, e.g., a murine monoclonal antibody and a human immunoglobulin constant region (CH1-CH2-CH3, CL). Chimeric antibodies and methods for their production are known in the art (Cabilly et al., 1984, Proc. Natl. Acad. Sci. USA 81:3273-3277; Morrison et al., 1984, Proc. Natl. Acad. Sci. USA 81:6851-6855; Boulianne et al., 1984, Nature 312:643-646; Cabilly et al., European Patent Application 125023 (published Nov. 14, 1984); Taniguchi et al., European Patent Application 171496 (published Feb. 19, 1985); Morrison et al., European Patent Application 173494 (published Mar. 5, 1986); Neuberger et al., PCT Application WO 86/01533 (published Mar. 13, 1986); Kudo et al., European Patent Application 184187 (published Jun. 11, 1986); Morrison et al., European Patent Application 173494 (published Mar. 5, 1986); Sahagan et al., 1986, J. Immunol. 137:1066-1074; Robinson et al., PCT/US86/02269 (published May 7, 1987); Liu et al., 1987, Proc. Natl. Acad. Sci. USA 84:3439-3443; Sun et al., 1987, Proc. Natl. Acad. Sci. USA 84:214-218; Better et al., 1988, Science 240:1041-1043). SMIPs are single-chain polypeptides comprising one binding domain, one hinge domain and one effector domain. SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

The antibodies which may be used according to the present invention may have one or several markers or labels. Such markers or labels may be useful to detect the antibody either in its diagnostic application or its therapeutic application. Preferably the markers and labels are selected from the group comprising avidin, streptavidin, biotin, gold and fluorescein and used, e.g., in ELISA methods. These and further markers as well as methods are, e.g., described in Harlow and Lane, supra.

In one embodiment, a PPRC antibody comprises a PKN3 activation-state-specific antibody, which recognizes the phospho-threonine at position 860 in the turn motif of PKN3 (boxed) (SEQ ID NO: 24: 847-YFEGEFTGLPPAL [symbol] PPAPHSLLTARQQA-874). Said antibody is useful inter alia as a probe for increased PKN3 expression and activation, and as a biomarker for patient stratification and therapeutic response.

A further class of medicaments, compounds that disrupt the PPRC, as well as diagnostic agents which may be generated using the PPRC complex or components and fragments thereof, or the nucleic acid encoding said PPRC complex or components and fragments thereof, are peptides which bind thereto. Such peptides may be generated by using methods according to the state of the art such as phage display. Basically, a library of peptides is generated and displayed on the surface of phage, and the displayed library is contacted with the target, in the present case, for example, the PPRC complex or components thereof. Those peptides binding to the target are subsequently removed, preferably as a complex with the target molecule, from the respective reaction. It is known to the skilled artisan that the binding characteristics, at least to a certain extent, depend on the particular experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterized.

Prior to the characterization step, an amplification step optionally may be performed such as, e.g., by propagating the peptide coding phages. In some embodiments, the characterization comprises the sequencing of the target binding peptides. Basically, the peptides are not limited in their lengths, however, peptides having a length from about 8 to 20 amino acids are generally obtained in the respective methods. The size of the libraries may be about 10² to 10¹⁸ or 10⁸ to 10¹⁵ different peptides, however, the size of the library is not limited thereto.

According to the present invention, the PPRC complex or components thereof, as well as the nucleic acids encoding said PPRC complex or components thereof, may be used as the target for the manufacture or development of a medicament for the treatment of an aggressive cancer, as well as for the manufacture or development of means for the diagnosis of said aggressive cancer in a screening process, whereby in the screening process small molecules or libraries of small molecules are used. This screening comprises the step of contacting the target PPRC complex or components thereof (target) with a single small molecule or a variety (such as a library) of small molecules at the same time or subsequently, preferably those from the library as specified above, and identifying those small molecules or members of the library which bind to the target and disrupt the function or integrity of the PPRC complex which, if screened in connection with other small molecules may be separated from the non-binding or non-interacting small molecules.

The binding and non-binding may strongly be influenced by the particular experimental set-up. In modifying the stringency of the reaction parameters, it is possible to vary the degree of binding and non-binding which allows a fine tuning of this screening process. In some embodiments, after the identification of one or several small molecules which specifically interact with the target, this small molecule may be further characterized. This further characterization may, for example, reside in the identification of the small molecule and determination of its molecular structure and further physical, chemical, biological or medical characteristics. In some embodiments, the natural compounds have a molecular weight of about 100 to 1000 Da. In some embodiments, small molecules are those which comply with Lepinski's Rule of Five, which is known to the skilled artisan (see Lipinski et al., Adv. Drug. Del. Rev., 23: 3-25, 1997). Alternatively, small molecules may also be defined such that they are synthetic-small-molecules arising from combinatorial chemistry, in contrast to natural products. However, it is to be noted that these definitions are only subsidiary to the general understanding of the respective terms in the art. Like all kinases, the PKN3 and PDK1 components of the PPRC complex contain an ATP-binding site and drugs that are known to bind to such sites are therefore suitable candidate compounds for inhibiting PPRC function. Examples of suitable compounds include, but are not limited to, LY-27632, Ro-3 1-8220, and HA 1077, all of which are available from Calbiochem (La Jolla, Calif.).

The invention is further exemplified by the following examples, which are not limiting of the scope of the invention.

### Example 1

### Constructs and Proteins

Flag-PKN3 protein: The full-length cDNA of human PKN3 (e.g., SEQ ID NO: 25 and SEQ ID NO: 26) was amplified by PCR and cloned into the pcDNA3 expression vector (SEQ ID NO: 27) (Invitrogen, Carlsbad, Calif.). The 5′ primer contained an ATG codon followed by a Flag epitope (SEQ ID NO: 28) in frame with the coding region that was amplified. This PCR product was digested with EcoRI and XhoI restriction enzymes and ligated into the pcDNA3 expression vector through the same enzyme sites to generate the N-terminal Flag epitope-tagged PKN3. Kinase dead version of PKN3, which comprises a K588R substitution, was also cloned into the same vector using the same strategy.

Myc-Rho proteins: The full-length cDNA of human Rac1 (e.g., SEQ ID NOs: 29 and 30), RhoA (e.g., SEQ ID NOs: 31 and 32), RhoB (e.g., SEQ ID NOs: 32 and 33) and RhoC (e.g., SEQ ID NOs: 35 and 36) were cloned into the pCG mammalian expression vector (see, Tanaka and Herr (1990) Cell, 60(3): 375-386). The 5′ primer contained an ATG codon followed by a Myc epitope (SEQ ID NO: 37) in frame with the coding region that was amplified. This PCR product was digested with NdeI and BamHI restriction enzymes and ligated into the pCG expression vector through the same enzyme sites to generate the N-terminal Myc epitope-tagged RhoA, RhoB and RhoC.

### Example 2

### Antibodies and Cell Lysis

Antibodies: p110 antibodies are described in Klippel et al, (1994) Mol Cell Biol., 14(4):2675-85. PKN antibodies have been described in Leenders, 2004. PDK1 antibodies are commercially available from Cell Signaling Technology, Inc. (Beverly, Mass.). Anti-phospho-PKN3 T860 rabbit monoclonal antibodies were produced according to standard procedures (see Spieker-Polet, 1995, Proc. Natl. Acad. Sci. USA, 92:9348-9352). Antibodies to the Myc epitope were the 9E10 Myc monoclonal antibodies, which are commercially available and are described in Evan et al., Mol. Cell. Biol., 5:3610-6 (1985).

Cell lysates: Cells were washed twice with cold phosphate-buffered saline (PBS) and lysed at 4° C. in lysis buffer containing 20 mM Tris (pH 7.5), 137 mM NaCl, 15% (vol/vol) glycerol, 1% (vol/vol) Nonidet P-40 (NP-40), 2 mM phenylmethylsulfonyl fluoride, 10 mg of aprotinin per ml, 20 mM leupeptin, 2 mM benzamidine, 1 mM sodium vanadate, 25 mM β-glycerolphosphate, 50 mM NaF, and 10 mM Na-pyrophosphate. Lysates were cleared by centrifugation at 14,000×g for 5 min, and aliquots of the lysates were analyzed for protein expression and enzyme activity (see below). Samples were separated by sodium dodecyl sulfate-polyacrylamide gel electrophoresis (SDS-PAGE) and transferred to nitrocellulose filters (Schleicher & Schuell). Filters were blocked in TBST buffer (10 mM Tris-HCl [pH 7.5], 150 mM NaCl, 0.05% [vol/vol] Tween 20, 0.5% [wt/vol] sodium azide) containing 5% (wt/vol) dried milk. The respective antibodies were added in TBST at appropriate dilutions. Bound antibody was detected with anti-mouse-, anti-goat, or anti-rabbit-conjugated alkaline phosphatase (Santa Cruz Biotechnology) in TBST, washed, and developed with nitroblue tetrazolium and 5-bromo-4-chloro-3-indolylphosphate (Promega). Alternatively, horseradish peroxidase-conjugated secondary antibodies were used and developed by enhanced chemiluminescence (Amersham).

### Example 3

### Expression Profile of PKN3 in Cancer Cells

It is known that increased PKN3 or phosphor-PKN3 (“P*-PKN3”) expression is associated with increased tumor metastasis (see Leenders et al., Biochem. Biophys. Res. Comm., 261:808-814). The expression of PKN3 and P*-PKN3 were examined in models of malignancy potential and drug resistance.

Breast cancer cell lines of increasing aggressiveness, including from least aggressive to most aggressive, MCF-10A, MCF7, MDA361, MCF468, BT549 and MDA231, were examined for PKN3 and P*-PKN3 expression. FIG. 1 shows a Western blot of extracts from those cells, probed for p110αPI3K, total PKN3 and phospho-PKN3 (P*-PKN3 T860). The results confirm that PKN3 and P*-PKN T860 are expressed to a larger extent in cancer cells having greater malignancy potential, than in those having lesser malignancy potential.

Non-small cell lung cancer (NSCLC) cell lines of increasing resistance to the anti-cancer drug gefitinib (a.k.a. IRESSA; 4-(3-chloro-4-fluoroanilino)-7-methoxy-6-(3-morpholino-propoxy)-quinazoline), including from least resistant to most resistant, H1650, H1975, A549, PC14, H157 and H460 cells, were examined for PKN3 and P*-PKN3 expression (see Noro et al., (2006) BMC Cancer, 6: 277-289). FIG. 2 shows a Western blot of extracts from those cells probed for (a) p110αPI3K, (b) total PKN3 and (c) P*-PKN3 T718. The results confirm that PKN3 and P*-PKN T860 are expressed to a larger extent in cancer cells having greater resistance to an anti-cancer drug, than in those having greater sensitivity to the anti-cancer drug.

### Example 4

### PPRC Complex Formation with Endogenous PKN3

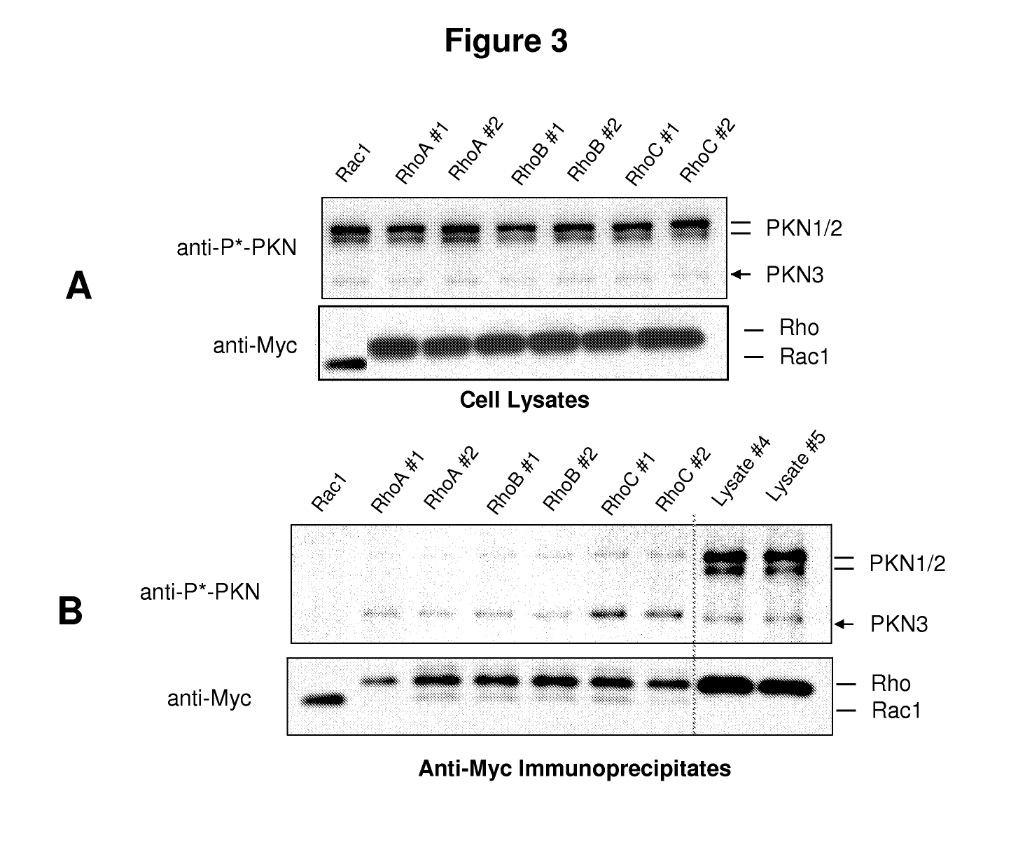

Immunoprecipitation experiments using cells transfected with Myc-tagged small GTPases Rac1, RhoA, RhoB or RhoC revealed that RhoC preferentially binds to phosphorylated PKN3 (FIG. 3, panel B). HeLa cells (5×10⁵ cells/10-cm tissue culture dish) were singly transfected with the indicated plasmids by FUGENE® transfection reagent (Roche Diagnostics, Indianapolis, Ind.). The immunoprecipitation was done with the 9E10 antibody to Myc conjugated with Protein G sepharose. The immunocomplex was washed three times with lysis buffer. The bound proteins were analyzed by Western blot using anti-P*-PKN3 T860 antibodies.

This experiment reveals that endogenous P*-PKN3 preferentially associates with the small GTPase RhoC.

### Example 5

### PPRC Complex Formation with Heterologous PKN3

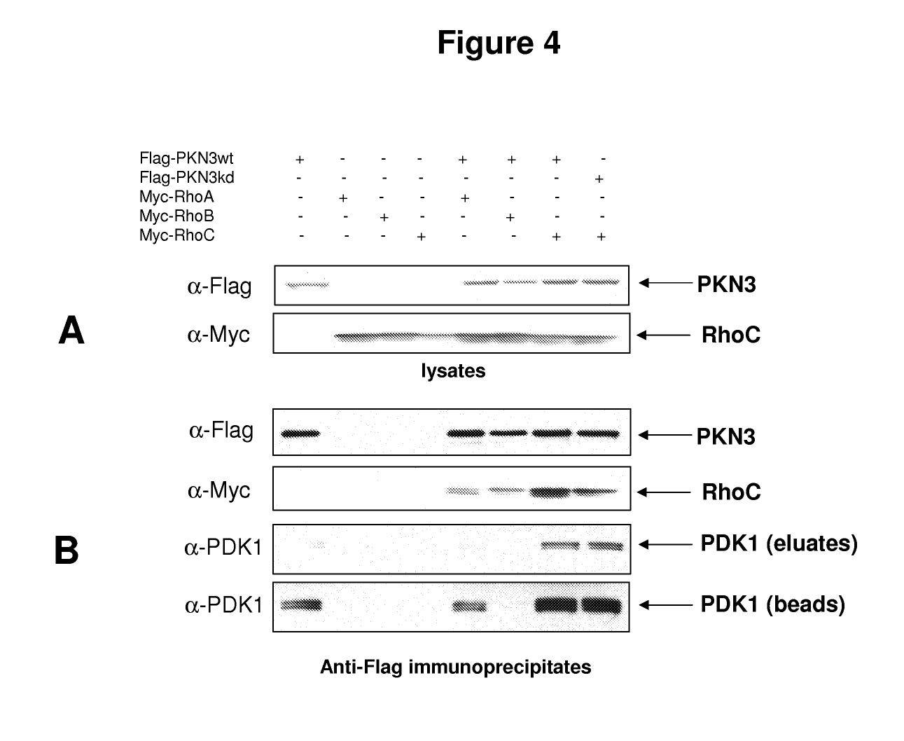

Immunoprecipitation experiments using HEK293T cells transfected with Flag-tagged PKN3 and Myc-tagged small GTPases RhoA, RhoB or RhoC revealed that PKN3 binds to phosphoinositide-dependent kinase-1 (PDK1) and RhoC. FIG. 4B indicates that the kinase-active form of PKN3 preferentially binds RhoC and PDK1. Thus, PKN3, RhoC and PDK1 form a ternary complex (“PPRC complex.”)

For immunoprecipitations with Flag (FIG. 4B), HEK293T cells (5×10⁵ cells/10-cm tissue culture dish) were either singly transfected or cotransfected with the indicated plasmids (Flag-PKN3 wt [full-length kinase active], Flag-PKN3 kd [kinase-dead PKN3], Myc-RhoA, Myc-RhoB or Myc-RhoC) by FUGENE® transfection reagent. After 6 hours of incubation at 37° C. in a 5% CO₂ incubator, cells were washed twice in serum-free Dulbecco's modified Eagle's medium (DMEM), and fresh medium (DMEM, 10% fetal bovine serum) was added to the cells for a further 48 h of incubation. Cells were washed with phosphate-buffered saline (PBS) and lysed in 0.5 ml of lysis buffer (50 mM Tris-HCl [pH 7.5], 150 mM NaCl, 10 mM β-glycerolphosphate, 10% glycerol, 1% Triton X-100, 1 mM Na₃VO₄, 1 mM NaF, and protease inhibitors [Roche Molecular Biochemicals]) for 30 minutes on ice. The cell lysates were centrifuged for 30 minutes at 14,000 rpm, and the supernatants from the spun lysates were incubated at 4° C. for overnight with anti-Flag M2 affinity resin (Sigma). The beads were then washed three times with TBS buffer (50 mM Tris-HCl [pH 7.5], 150 mM NaCl), and bound proteins were eluted with TBS buffer containing 10% glycerol, 1 mM DTT and 200 μg of Flag peptide (Sigma)/ml. The bound products were visualized by Western blot analysis (FIG. 4).

### Example 6

### PPRC Complex Kinase Activity

The enzymatic activity of the PPRC complex was examined by assessing (i) the phosphorylation status of the PKN3 component of the PPRC complex, and (ii) the kinase activity of the PPRC toward a substrate, i.e., a GSK3α-derived fragment having a sequence of GPGRRGRRRTSSFAEGG (SEQ ID NO:1).

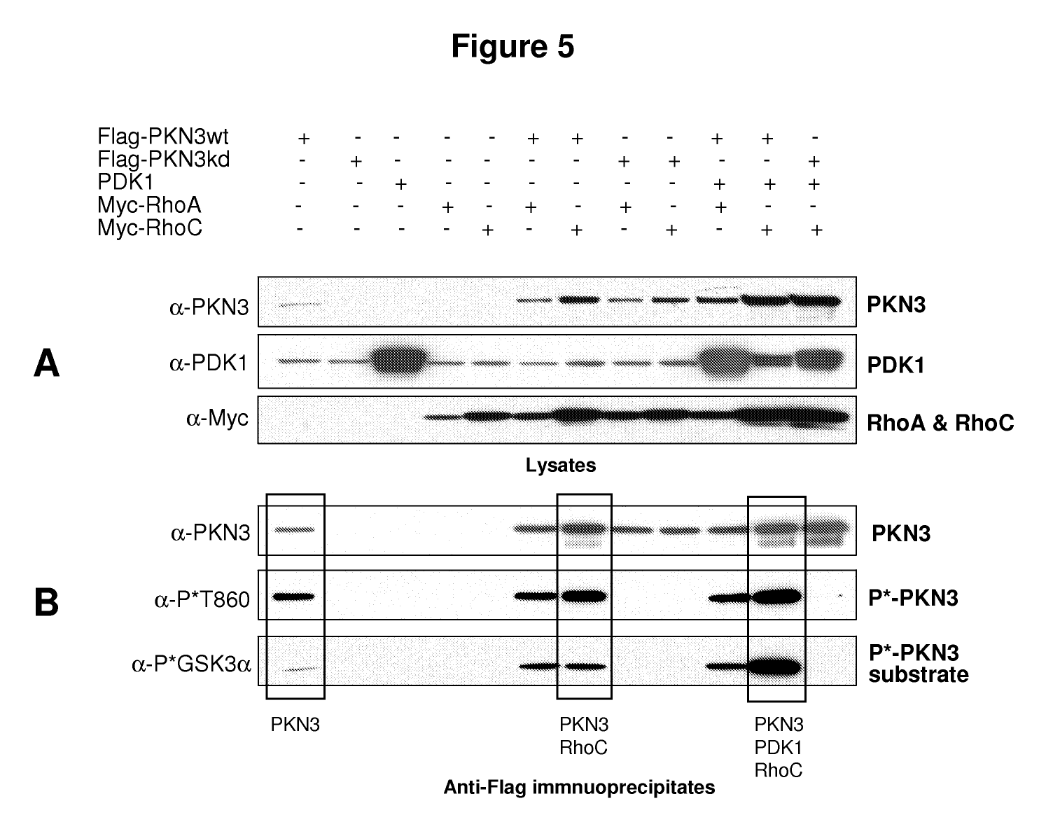

Immunoprecipitation experiments using PC3 cells transfected with Flag-tagged PKN3 and Myc-tagged small GTPases RhoA, RhoB or RhoC revealed that PKN3 binds to phosphoinositide-dependent kinase-1 (PDK1) and RhoC. FIG. 5B indicates that the kinase activity is greatest for the PPRC complex than either PKN3 alone, or PKN3 and RhoC combined.

For Flag-immunoprecipitations followed by a kinase assay, PC3 cells (5×10⁵ cells/10-cm tissue culture dish) were either singly transfected or cotransfected with the indicated plasmids by FUGENE® transfection reagent as described above. Cells were washed with phosphate-buffered saline (PBS) and lysed in 0.3% Triton X-100 in the washing buffer (20 mM Tris-HCl (pH 7.5), 1 mM EDTA, 1 mM EGTA, 5 mM MgCl2, 1 uM okadaic acid, 10 mM glycerolphosphate, 25 mM sodium fluoride, 5 mM sodium pyrophosphate, 0.2 mM PMSF, and 2 mM DTT). Immunoprecipitation was done with anti-Flag M2 affinity resin (Sigma). The immunocomplex was washed three times with the above buffer containing 0.5 M NaCl and twice with the kinase buffer (20 mM Tris-HCl (pH 7.5), 10 mM MgCl₂, 1 mM EDTA, and 1 mM okadaic acid) containing β-glycerolphosphate and 10 mM sodium pyrophosphate. The complex was finally suspended in the kinase buffer and incubated with 50 μM ATP and PKN3 substrate at RT for 15 minutes. Phosphorylated proteins were separated on SDS-polyacrylamide gels, visualized by Western blotting.

### Example 7

### IN Vivo Prostate Cancer Model

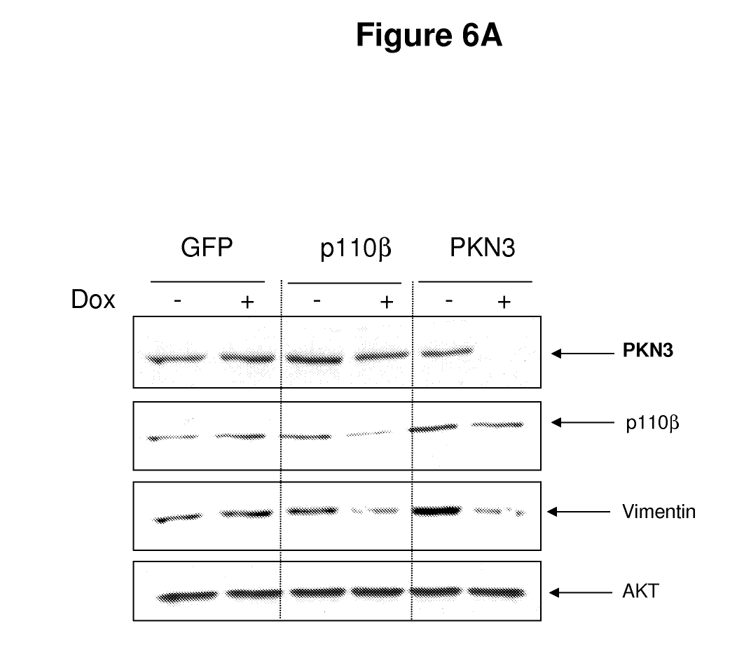

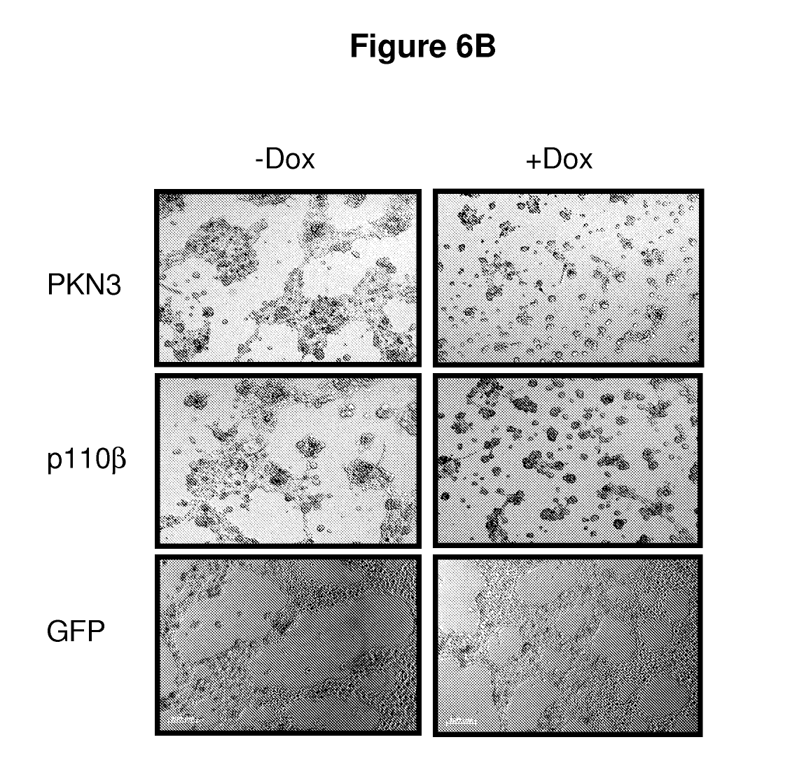

PC-3 (prostate cancer) cells were engineered to express shRNAs targeting PKN3 and p110β. Doxycycline (Dox)-induced shRNA expression resulted in efficient knockdown of PKN3 and p110β protein levels after 72 h (FIG. 6A). Inhibition of both PKN3 and p110β resulted in reduction of the vimentin protein level, which is a well known marker for epithelial-mesenchymal transition (EMT) (FIG. 6A). Respective cell populations were seeded on MATRIGEL and cells with shRNA-mediated knockdown of PKN3 expression exhibited impaired growth on extracellular matrix. Since p110β represents the predominant PI3K subunit in PC-3 cells, inhibition of p110β also interfered with MATRIGEL growth of PC-3 cells, and served as a positive control (FIG. 6B)

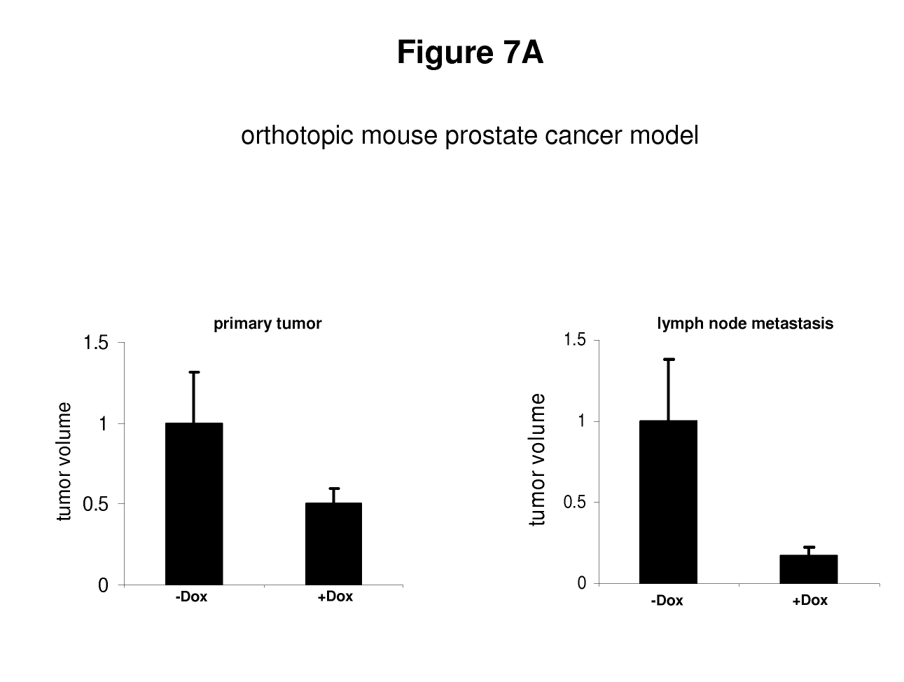

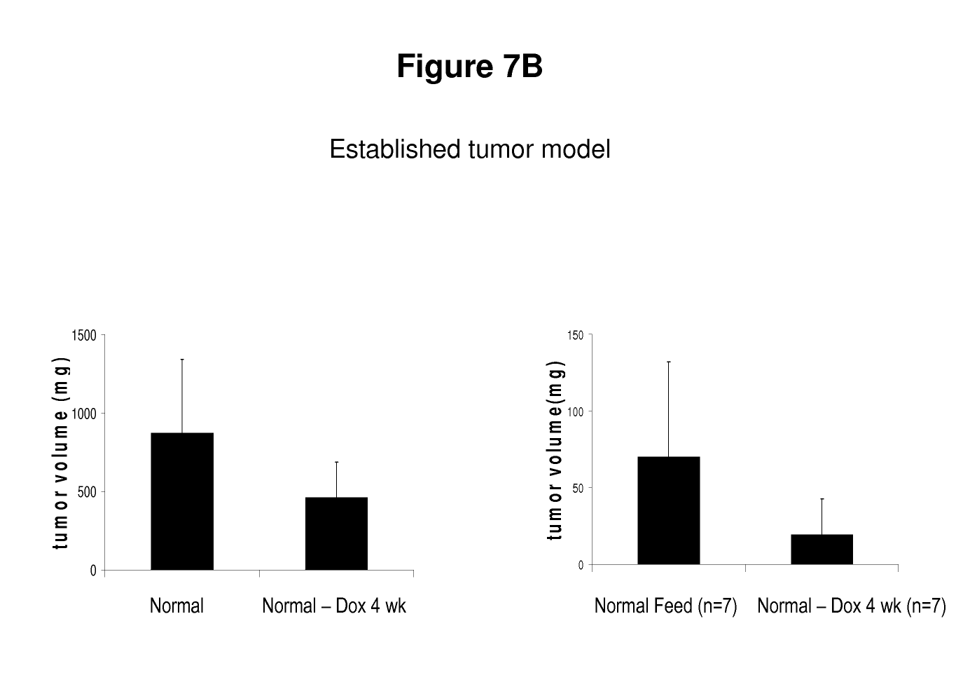

Next, stable PKN3 shRNA PC-3 cells were transplanted intraprostatically into nude mice. The animals were split into two groups, one group was treated with Dox to induce shRNA expression and the second group was mock-treated. After 49 days the mice were killed and analyzed for primary tumor formation and lymph node metastasis. The Dox-treated group of mice showed a strong effect on primary tumor growth and metastases formation was also strongly inhibited (FIG. 7A).

PKN3 was also validated in established prostate tumors. In this experiment, all the animals were kept on a normal diet for 4 weeks allowing the cells to form the primary tumor before PKN3 is silenced by a Dox diet. After 4 weeks of normal diet, inhibition of PKN3 interfered with tumor growth and metastasis in an established tumor model (FIG. 7B).

### Example 8

### IN Vivo Breast Cancer Model

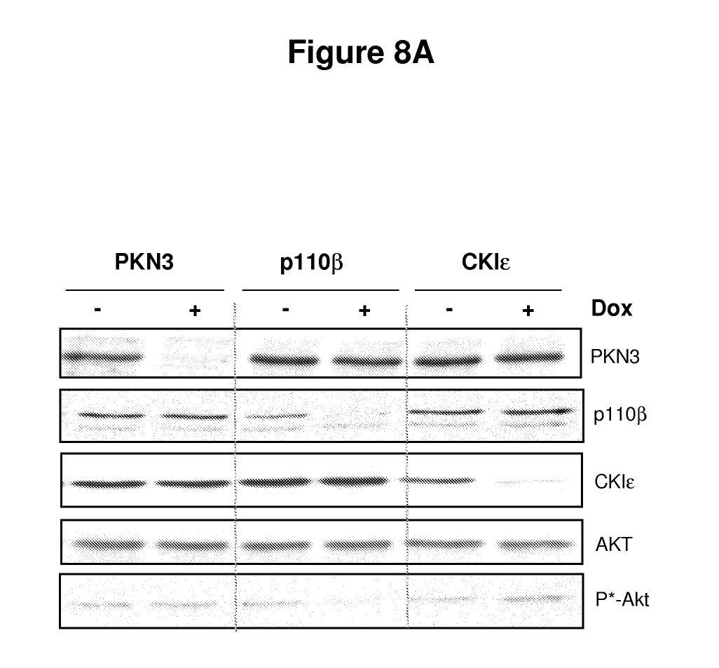

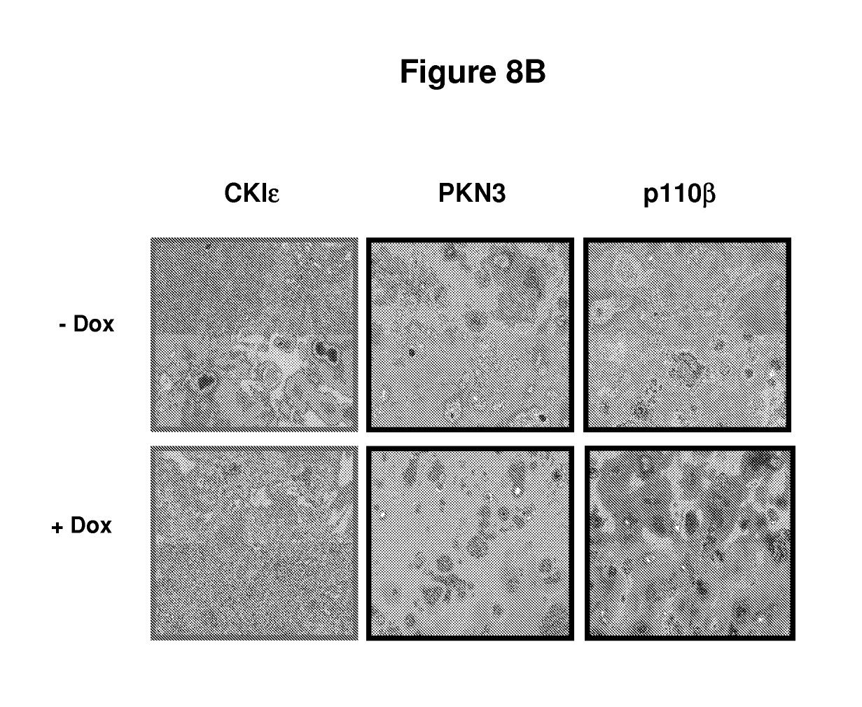

MDA-MB-231 (breast cancer) cells were engineered to express shRNAs targeting PKN3, p110β, and CKIε. Dox-induced shRNA expression resulted in efficient knockdown of PKN3, p110β and CKIε protein levels after 72 h (FIG. 8A). Respective cell populations were seeded in MATRIGEL and cells with shRNA-mediated knockdown of PKN3 expression exhibited impaired growth in the extracellular matrix, which appeared to be stronger than the knockdown of p110β. Accordingly, induced inhibition of CKIε had no effect in MDA-MB-231 cells in this experiment and served as a control for Dox treatment and shRNA induction (FIG. 8B).

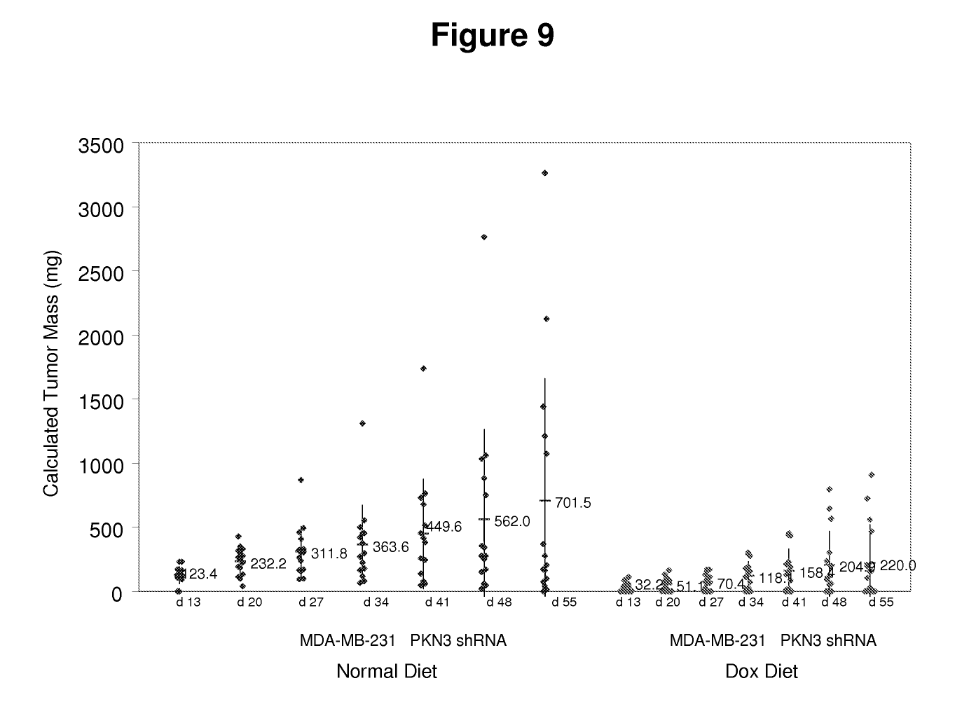

An orthotopic breast cancer model was generated using the same engineered cell line to do in vivo validation of PKN3 in breast cancer. Stable PKN3 shRNA MDA-MB-231 cells were grown in the absence of Dox and injected into the mammary fat pad of animals. The animals were split into two groups: one group was treated with Dox to induce shRNA expression and the second group was mock-treated. The size of the tumors were measured weekly until day 55. The Dox-treated group of mice showed a strong effect on breast tumor growth as shown in FIG. 9.

What is claimed is:

1. A method of identifying a compound useful in the treatment of cancer, the method comprising:
a. contacting a test cell with a test compound, the cell is ex vivo and comprises of PKN3 polypeptide or fragment thereof, a RhoC polypeptide or fragment thereof, and a PDK1 polypeptide or a fragment thereof;
b. determining a test level of a complex, which is formed in the presence of the test compound and which comprises the PKN3 polypeptide, the RhoC polypeptide and the PDK1 polypeptide; and
c. comparing the test level determined in step (b) to a reference,
wherein a difference between the test level determined in step (b) and the reference in step (c) indicates that the test compound has cancer therapeutic potential.

2. The method of claim 1, wherein the cell is selected from the group consisting of a PC3 cell, a HEK293 cell, A MDA-MB231 cell and a HeLa cell.

3. The method of claim 1, wherein the PKN3 polypeptide comprises a molecular tag selected from the group consisting of FLAG tag, GST tag and Myc tag, and the complex is isolated by pulling down the PKN3 polypeptide by its molecular tag.

4. The method of claim 1, wherein the reference is either (a) a level of a complex comprising a PKN3 polypeptide and a RhoC polypeptide that is formed in the absence of the test compound or (h) a complex comprising a PKN3 polypeptide and a RhoC polypeptide that is formed in the presence of the test compound, wherein the PKN3 polypeptide or the PDK1 polypeptide is kinase dead.

5. A method of diagnosing cancer in a patient comprising:
a. obtaining a sample from a patient;
b. determining a level of PKN3 activity and a level of RhoC activity in the sample, thereby generating a test level; and
c. comparing the test level to a reference,
wherein a difference between the test level and the reference indicates the likelihood that the cancer is aggressive.

6. A method of identifying a patient who can respond to a cancer therapy comprising:
a. obtaining a sample from a patient:
b. determining a level of PKN3 activity and a level of RhoC activity in the sample, thereby generating a test level; and
c. comparing the test level to a reference,
wherein a difference between the test level and the reference indicates the likelihood that the patient will respond to the cancer therapy.

7. (canceled)

8. (canceled)

9. A method for determining the efficacy of a cancer therapeutic treatment regimen in a subject, comprising:
a. obtaining a first sample from a patient;
b. determining a level of PKN3 activity and a level of RhoC activity in the first sample, thereby generating a first level;
c. administering the treatment regimen to the subject;
d. obtaining a second sample from the patient following the administration of the treatment regimen;
e. determining a level of PKN3 activity and a level of RhoC activity in the second sample, thereby generating a second level; and
f. comparing the first and second levels,
wherein a decrease in both PKN3 activity and RhoC activity in the second level relative to the first level indicates that the treatment regimen is effective against a cancer in the patient.

10. (canceled)

11. (canceled)

12. (canceled)

13. The method of claim 1, wherein the PNK3 polypeptide comprises either (a) an amino acid sequence that is at least 95% identical to SEQ ID NO:26, or (h) an amino acid sequence that is at least 95% identical to SEQ 1D NO:36.

14. (canceled)

15. A polypeptide comprising a complementarity determining region (CDR) that binds to a PKN3 turn motif phosphorylation site at T860.

16. (canceled)

17. (canceled)

18. (canceled)

19. A polypeptide that inactivates a PPRC complex, wherein
a. the PPRC complex comprises a PKN3 polypeptide, a RhoC polypeptide, and a PDK1 polypeptide,
b. the polypeptide binds to (i) a domain of a RhoC polypeptide that binds to a cognate domain of a PKN3 polypeptide, or (ii) a domain of a PKN3 polypeptide that binds to a cognate domain of a RhoC polypeptide, and
c. the polypeptide (i) inhibits the formation of the PPRC complex or (ii) inhibits the kinase activity associated with the PPRC complex.

20. The polypeptide of claim 19, wherein the polypeptide comprises at least one ACC domain selected from the group consisting of ACC1, ACC2 and ACC3 of a PKN3 and does not comprise a kinase domain of PKN3.


SEQUENCE LISTING (37 sequences)

SEQ ID NO: 1 - protein, 17 aa, Homo sapiens
GPGRRGRRRTSSFAEGG

SEQ ID NO: 2 - protein, 30 aa, Homo sapiens
RRAIPTVNHSGTFSPQAPVPTTVPVVDVRI

SEQ ID NO: 3 - protein, 33 aa, Homo sapiens
KEGMGYGDRTSTFCGTPEFLAPEVLTETSYTRA

SEQ ID NO: 4 - protein, 13 aa, Homo sapiens
RRGPSPPASPTRK

SEQ ID NO: 5 - protein, 12 aa, Homo sapiens
KGCPRTPTTLRE

SEQ ID NO: 6 - protein, 26 aa, Homo sapiens
KGCPRTPTTLREASDPATPSNFLPKK

SEQ ID NO: 7 - protein, 33 aa, Homo sapiens
KEGIGFGDRTSTFCGTPEFLAPEVLTQEAYTRA

SEQ ID NO: 8 - protein, 31 aa, Homo sapiens
KERPISMINEASNYNVTSDYAVHPMSPVGRT

SEQ ID NO: 9 - protein, 18 aa, Homo sapiens
RTNPPTQKPPSPPMSGRG

SEQ ID NO: 10 - protein, 27 aa, Homo sapiens
KKEEENADSDDEGELQDLLSQDWRVKG

SEQ ID NO: 11 - protein, 25 aa, Homo sapiens
RTSSPRSPPSSSEIFTPAHEENVRF

SEQ ID NO: 12 - protein, 29 aa, Homo sapiens
KFIVLNSLNQNAKPEGPEQAELGRLSPRA

SEQ ID NO: 13 - protein, 20 aa, Homo sapiens
KGGGGYTCQSGSGWDEFTKH

SEQ ID NO: 14 - protein, 10 aa, Homo sapiens
RLTEGCSFRR

SEQ ID NO: 15 - protein, 27 aa, Homo sapiens
RTNTNVNCPIECFMPLDVQADREDSRE

SEQ ID NO: 16 - protein, 20 aa, Homo sapiens
KLPVGSQCSVDLESASGEKD

SEQ ID NO: 17 - protein, 13 aa, Homo sapiens
KIEDSEENGVFKY

SEQ ID NO: 18 - protein, 17 aa, Homo sapiens
KAAREYEDPPSEEEDKI

SEQ ID NO: 19 - protein, 15 aa, Homo sapiens
KFIIPGSPAIISIRQ

SEQ ID NO: 20 - protein, 35 aa, Homo sapiens
KDSLSVSSNDASPPASVASLQPHMIGAQSSPGPKR

SEQ ID NO: 21 - protein, 18 aa, Homo sapiens
RRSTQGVTLTDLKEAEKA

SEQ ID NO: 22 - protein, 33 aa, Homo sapiens
KEFGSMVLELNASDDRGIDIIRGPILSFASTRT

SEQ ID NO: 23 - protein, 21 aa, Homo sapiens
KESKDKPEIEDVGSDEEEEKK

SEQ ID NO: 24 - protein, 28 aa, Homo sapiens
YFEGEFTGLPPALTPPAPHSLLTARQQA

SEQ ID NO: 25 - DNA, 3385 bp, Homo sapiens
ggctcccgcgggcgcgcggcggggaaggccagaggacctgggcgcgggcgatgtgcctcctgagcgtccaaaccgggggtgaggcgcggtcacgcccagcgggaaccgcaggcgccgaagcccgggtactgggcccagaatcccgcggaattttggatccgagggaggcgctggggcgcgggacctcgggcgtggggtcccgggcgctggatcggcgcggacgggaggcggcgctggtcccgcgggccagcgggtctcgggagggggcgcccgatcccgcgtctccggcgccgcttcccgggaagtttcaagtttgaaagtcctggcggagggtctgcggcttccgggaccggagtggctgagaggagggccccaagcggccggagcggcgccatggaggagggggcgccgcggcagcctgggccgagccagtggcccccagaggatgagaaggaggtgatccgccgggccatccagaaagagctgaagatcaaggagggggtggagaacctgcggcgcgtggccacagaccgccgccacttgggccatgtgcagcagctgctgcggtcctccaaccgccgcctggagcagctgcatggcgagctgcgggagctgcacgcccgaatcctgctgcccggccctgggcctggcccagctgagcctgtggcctcaggaccccggccgtgggcagagcagctcagggctcggcacctagaggctctccggaggcagctgcatgtggagctgaaggtgaagcagggggctgagaacatgacccacacgtgcgccagtggcacccccaaggagaggaagctcctggcagctgcccagcagatgctgcgggacagccagctgaaggtggccctgctgcggatgaagatcagcagcctggaggccagtgggtccccggagccagggcctgagctgctggcggaggagctacagcatcgactgcacgttgaggcagctgtggctgagggcgccaagaacgtggtgaaactgcttagtagccggagaacacaggaccgcaaggcactggctgaggcccaggcccagctacaggagtcctctcagaaactggacctcctgcgcctggccttggagcagctgctggagcaactgcctcctgcccaccctttgcgcagcagagtgacccgagagttgcgggctgcggtgcctggatacccccagccttcagggacacctgtgaagcccaccgccctaacagggacactgcaggtccgcctcctgggctgtgaacagttgctgacagccgtgcctgggcgctccccagcggccgcactggccagcagcccctccgagggctggcttcggaccaaggccaagcaccagcgtggccgaggcgagcttgccagcgaggtgctggctgtgctaaaggtggacaaccgtgttgtggggcagacgggctgggggcaggtggccgaacagtcctgggaccagacctttgtcatcccactggagcgagcccgtgagctggagattggggtacactggcgggactggcggcagctatgtggcgtggccttcctgagacttgaggacttcctggacaatgcctgtcaccaactgtccctcagcctggtaccgcagggactgctttttgcccaggtgaccttctgcgatcctgtcattgagaggcggccccggctgcagaggcaggaacgcatcttctctaaacgcagaggccaggacttcctgagggcttcgcagatgaacctcggcatggcggcctgggggcgcctcgtcatgaacctgctgcccccctgcagctccccgagcacaatcagcccccctaaaggatgccctcggaccccaacaacactgcgagaggcctctgaccctgccactcccagtaatttcctgcccaagaagacccccttgggtgaagagatgacacccccacccaagcccccacgcctctacctcccccaggagccaacatccgaggagactccgcgcaccaaacgtccccatatggagcctaggactcgacgtgggccatctccaccagcctcccccaccaggaaaccccctcggcttcaggacttccgctgcttagctgtgctgggccggggacactttgggaaggtcctcctggtccagttcaaggggacagggaaatactacgccatcaaagcactgaagaagcaggaggtgctcagccgggacgagatagagagcctgtactgcgagaagcggatcctggaggctgtgggctgcacagggcaccctttcctgctctccctccttgcctgcttccagacctccagccatgcctgctttgtgactgagtttgtgcctggtggtgacctcatgatgcagatccacgaggatgtcttccccgagccccaggcccgcttctacgtggcttgtgttgtcctggggctgcagttcttacacgagaagaagatcatttacagggacctgaagttggataaccttctgctggatgcccagggattcctgaagatcgcagactttggactctgcaaggaagggatcggcttcggggaccggactagcaccttctgtggcaccccggagttcctggctcccgaggtgctgacccaggaggcatacacacgggctgtggactggtgggggctgggtgtgctgctctacgagatgctggtgggtgagtgcccgttcccaggggacacagaggaagaggtgtttgactgcatcgtcaacatggacgccccctaccccggctttctgtcggtgcaagggcttgagttcattcagaagctcctccagaagtgcccggagaagcgcctcggggcaggtgagcaggatgccgaggagatcaaggtccagccattcttcaggaccaccaactggcaagccctgctcgcccgcaccatccagccccccttcgtgcctaccctgtgtggccctgcggacctgcgctactttgagggcgagttcacagggctgccgcctgccctgaccccacctgcaccccacagcctcctcactgcccgccaacaggccgccttccgggacttcgactttgtgtcagagcgattcctggaaccctgagggcatctcctggcacctctgtccccttcccccacagactgttagagcctctgctcgttcacccgtgcgccctgcctggaggtccaggccttgctgggtacttctgagcccttgggattcaaagtggcagccatggggccactgttgtgggctttgctcagtgtcactgggcaaagtgtgtcccttccccctccagctcgccctcttctacctcccagcgagacctggcccagaaagggtgccgcagcaaggagtgatatggtttgtctttttaagactggacttgctttatattaaatttgtaaaagtgtgcaaaaaaaaa

SEQ ID NO: 26 - protein, 889 aa, Homo sapiens
MEEGAPRQPGPSQWPPEDEKEVIRRAIQKELKIKEGVENLRRVATDRRHLGHVQQLLRSSNRRLEQLHGELRELHARILLPGPGPGPAEPVASGPRPWAEQLRARHLEALRRQLHVELKVKQGAENMTHTCASGTPKERKLLAAAQQMLRDSQLKVALLRMKISSLEASGSPEPGPELLAEELQHRLHVEAAVAEGAKNVVKLLSSRRTQDRKALAEAQAQLQESSQKLDLLRLALEQLLEQLPPAHPLRSRVTRELRAAVPGYPQPSGTPVKPTALTGTLQVRLLGCEQLLTAVPGRSPAAALASSPSEGWLRTKAKHQRGRGELASEVLAVLKVDNRVVGQTGWGQVAEQSWDQTFVIPLERARELEIGVHWRDWRQLCGVAFLRLEDFLDNACHQLSLSLVPQGLLFAQVTFCDPVIERRPRLQRQERIFSKRRGQDFLRASQMNLGMAAWGRLVMNLLPPCSSPSTISPPKGCPRTPTTLREASDPATPSNFLPKKTPLGEEMTPPPKPPRLYLPQEPTSEETPRTKRPHMEPRTRRGPSPPASPTRKPPRLQDFRCLAVLGRGHFGKVLLVQFKGTGKYYAIKALKKQEVLSRDEIESLYCEKRILEAVGCTGHPFLLSLLACFQTSSHACFVTEFVPGGDLMMQIHEDVFPEPQARFYVACVVLGLQFLHEKKIIYRDLKLDNLLLDAQGFLKIADFGLCKEGIGFGDRTSTFCGTPEFLAPEVLTQEAYTRAVDWWGLGVLLYEMLVGECPFPGDTEEEVFDCIVNMDAPYPGFLSVQGLEFIQKLLQKCPEKRLGAGEQDAEEIKVQPFFRTTNWQALLARTIQPPFVPTLCGPADLRYFEGEFTGLPPALTPPAPHSLLTARQQAAFRDFDFVSERFLEP

SEQ ID NO: 27 - DNA, 5446 bp, Artificial
  synthetic plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 28 - protein, 7 aa, Artificial
  synthetic epitope
DYKDDDK

SEQ ID NO: 29 - DNA, 2341 bp, Homo sapiens
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcata

SEQ ID NO: 30 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 31 - DNA, 1926 bp, Homo sapiens
gtggatgagctgtgagtgcgcgcgcgtgcgcggggccgcgacctgtgccggctcgagcccgctgggcactcggaggcgcgcacgtcgttccccgccctcccgccgccgcccgccctcgctctctcgcgctaccctcccgccgcccgcggtcctccgtcggttctctcgttagtccacggtctggtcttcagctacccgccttcgtctccgagtttgcgactcgcggaccggcgtccccggcgcgaagaggctggactcggattcgttgcctgagcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcattttttaaattcccgttttgtcactttttctgataaaagatggccatattaccccttttcggccccatgtatctcagtaccccatggagctgggctaagtaaataggaattggtttcacgcctgaggcaattagacactttggaagatggcataacctgtctcacctggacttaagcatctggctctaattcacagtgctcttttctcctcactgtatccaggttccctcccagaggagccaccagttctcatgggtggcactcagtctctcttctctccagctgactaaactttttttctgtaccagttaatttttccaactactaatagaataaaggcagttttctaaaaaaa

SEQ ID NO: 32 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 33 - DNA, 2384 bp, Homo sapiens
atctgccaccgcagtctggttggagctgttgtcttgtatgctcagcgaggcccggagagacccgggagagagctaggccgagtccaccgcccgagtctgctgcccgagcccgcgttacgcacaaagccgccgatccccggcctggggtgagcagagcgaccaccgcccgggagcagcgcggcgagacgcacggtgcgccctatgcccccgcgcccccaccgcccccgccgcggcagccgaagcgcagcgagagaacgcgccaccgcggggcccgggtgcagctagcgaccctctcgccacctgcgcgcagcccgaggtgagcagtgagcggcgagcgggagggcagcgaggcgttcgcgggccccctcctgctgcccgggcccggcccgctcatggcggccatccgcaagaagctggtggtggtgggcgacggcgcgtgtggcaagacgtgcctgctgatcgtgttcagtaaggacgagttccccgaggtgtacgtgcccaccgtcttcgagaactatgtggccgacattgaggtggacggcaagcaggtggagctggcgctgtgggacacggcgggccaggaggactacgaccgcctgcggccgctctcctacccggacaccgacgtcattctcatgtgcttctcggtggacagcccggactcgctggagaacatccccgagaagtgggtccccgaggtgaagcacttctgtcccaatgtgcccatcatcctggtggccaacaaaaaagacctgcgcagcgacgagcatgtccgcacagagctggcccgcatgaagcaggaacccgtgcgcacggatgacggccgcgccatggccgtgcgcatccaagcctacgactacctcgagtgctctgccaagaccaaggaaggcgtgcgcgaggtcttcgagacggccacgcgcgccgcgctgcagaagcgctacggctcccagaacggctgcatcaactgctgcaaggtgctatgagggccgcgcccgtcgcgcctgcccctgccggcacggctccccctcctggaccagtcccccgcgagcccggagaaggggagacccgtgtcccacaaggaccccaccggcctgcctggcatctgtctgctgacgcctctggcttgcgccaggacttggcgtgggcaccgggcgcccccatcccagtgtctgtgtgcgtccagctgtgttgcacaggcctgggctccccactgagtgccaagggtcccctgagcatgcttttctgaagagccgggcctcagagtgtgtggctgtgtgtctgttcgactcccctcgccccattttcaccccacccccgcctctgatccccgggggcgagattggcgcgggagtgtggccgcgccccatcagatgttctcccttcaccagcgggagcttgatatcccttgtctgtaacatagaccccgggtactgcgggaggggagggctgctggggaggatggggggatgttatataaatatagatataattttattttcggagctaagatggtgttatttaagggtggtgatgggtgagcgctctggcccaggctgggccagactcccgcccaagcatgaacaggacttgaccatctttccaacccctggggaagacatttgcaactgacttggggaggacacagcttcagcacagcctctcctgcgggccagcccgctgcgaaccctccaccagctaccggagggaggagggaggatgcgctgtggggttgtttttgccataagcgaactttgtgcctgtcctagaagtgaaaattgttcagtccaagaaactgatgttatttgatttatttaaaggctaaaatttgtttttttattctttgcacaattgtttcattgtttgacacttaatgcactcgtcatttgcatacgacagtagcattctgaccacacttgtacgctgtaacctcatctacttctgatgtttttaaaaaatgacttttaacaaggagagggaaaagaaacccactaaattttgctttgtttccttgaagaatgtggcaacactgttttgtgattttatttgtgcaggtcatgcacacagttttgataaagggcagtaacaagtattggggcctattttttttttttccacaaggcattctctaaagctatgtgaaattttctctgcacctctgtacagagaatacacctgcccctgtatatccttttttcccctcccctccctcccagtggtacttctactaaattgttgtcttgttttttattttttaaataaactgacaaatgacaaaatggtgagcttatgatgtttacataaaagttctataagctgtgtatacagttttttatgtaaaatattaaaagactatgatgatgacatttaaaaaaaaaaaaaaaaaa

SEQ ID NO: 34 - protein, 196 aa, Homo sapiens
MAAIRKKLVVVGDGACGKTCLLIVFSKDEFPEVYVPTVFENYVADIEVDGKQVELALWDTAGQEDYDRLRPLSYPDTDVILMCFSVDSPDSLENIPEKWVPEVKHFCPNVPIILVANKKDLRSDEHVRTELARMKQEPVRTDDGRAMAVRIQAYDYLECSAKTKEGVREVFETATRAALQKRYGSQNGCINCCKVL

SEQ ID NO: 35 - DNA, 1346 bp, Homo sapiens
attgaaggctgggcagagtctgagtccacccgggtcgtgctccccccgctcgcccggctcctccgcagtccaggaatctccccgtggctctccccgacctggaggggtggacgcccctggcccccagtccccggcctgcggagggggccggtggctgcggccctgcgcggggccggggcgggccgagccaagggccgcccccggccgaccctccccctgccgggcccgccctccccgccgcggcgctggaggagggcggggcggggccctggggtcagtctgagcctccggcaccggccgcgcagctggaggcggcggagcggaagccccaccatggctgcaatccgaaagaagctggtgatcgttggggatggtgcctgtgggaagacctgcctcctcatcgtcttcagcaaggatcagtttccggaggtctacgtccctactgtctttgagaactatattgcggacattgaggtggacggcaagcaggtggagctggctctgtgggacacagcagggcaggaagactatgatcgactgcggcctctctcctacccggacactgatgtcatcctcatgtgcttctccatcgacagccctgacagcctggaaaacattcctgagaagtggaccccagaggtgaagcacttctgccccaacgtgcccatcatcctggtggggaataagaaggacctgaggcaagacgagcacaccaggagagagctggccaagatgaagcaggagcccgttcggtctgaggaaggccgggacatggcgaaccggatcagtgcctttggctaccttgagtgctcagccaagaccaaggagggagtgcgggaggtgtttgagatggccactcgggctggcctccaggtccgcaagaacaagcgtcggaggggctgtcccattctctgagatccccaaggcctttcctacatgccccctcccttcacaggggtacagaaattatccccctacaaccccagcctcctgagggctccatgctgaaggctcccattttcagttccctcctgcccaggactgcattgttttctagccccgaggtggtggcacgggccctccctcccagcgctctgggagccacgcctatgccctgcccttcctcagggcccctggggatcttgccccctttgaccttccccaaaggatggtcacacaccagcactttatacacttctggctcacaggaaagtgtctgcagtaggggacccagagtcccaggcccctggagttgttttcggcaggggccttgtctctcactgcatttggtcaggggggcatgaataaaggctacaggctccaacgtgaaaaaaaaaaaaaaaaaa

SEQ ID NO: 36 - protein, 193 aa, Homo sapiens
MAAIRKKLVIVGDGACGKTCLLIVFSKDQFPEVYVPTVFENYIADIEVDGKQVELALWDTAGQEDYDRLRPLSYPDTDVILMCFSIDSPDSLENIPEKWTPEVKHFCPNVPIILVGNKKDLRQDEHTRRELAKMKQEPVRSEEGRDMANRISAFGYLECSAKTKEGVREVFEMATRAGLQVRKNKRRRGCPIL

SEQ ID NO: 37 - protein, 10 aa, Artificial
  synthetic epitope
EQKLISEEDL